SEQUENCE LISTING (2 sequences)

SEQ ID NO: 1 - DNA, 19 bp, Artificial Sequence
  Synthetic
tataaaacccggcggcgca

SEQ ID NO: 2 - DNA, 20 bp, Artificial Sequence
  Synthetic
tcatccatggcgaactggtg

(19) United States
(12) Patent Application Publication
(10) Pub. No.: US 2024/0218350 A1
(43) Pub. Date: Jul. 4, 2024

(54) METHOD FOR ISOLATING AND PURIFYING NUCLEIC ACID SOLID FROM BIOLOGICAL MATERIAL

(71) Applicant: TIANJIN SPRING TIDE GENE TECHNOLOGY CO., LTD., Tianjin (JP)

(72) Inventor: Xianglong YANG, Tianjin (CN)

(73) Assignee: TIANJIN SPRING TIDE GENE TECHNOLOGY CO., LTD, Tianjin (CN)

(21) Appl. No.: 18/028,746

(22) PCT Filed: Sep. 28, 2021
(86) PCT No.: PCT/CN2021/121371

§ 371 Date: Mar. 27, 2023

(30) Foreign Application Priority Data
Sep. 30, 2020 (CN) 202011063801.4

(51) Int. Cl. C12N 15/10

(52) U.S. Cl. CPC C12N 15/1003

(57) ABSTRACT

The present invention discloses a method for isolating and purifying solid nucleic acids from biomaterials comprising the steps of (1) mixing the liquid containing naked nucleic acids with a precipitant; centrifuging at room temperature, pouring the supernatant into a new centrifuge tube; (2) adding isopropanol to the new centrifuge tube, mixing well, standing at room temperature, centrifuging, producing a solid, discarding other than the solid; washing, obtaining solid RNAs or solid mixtures of RNAs and DNAs and storing. The invention uses a homogenization-dissociation reagent to homogenize biomaterials, and a liquid containing naked nucleic acids can be easily obtained without heating, which can be used to extract solid RNAs and solid DNAs. By adding a compound that removes secondary metabolites of cells to the homogenization-dissociation reagent, vascular plant RNA, woody plant RNA, and both solid RNAs and solid DNA can be extracted from human blood. The solid RNAs and solid DNAs obtained by the present invention can be stored at room temperature for one month or below −20° C. for one year.

### TECHNICAL FIELD OF THE INVENTION

The present invention relates to methods for extracting out solid RNAs or (and) solid DNAs from biomaterials, including animal organs, animal tissues, animal cells, plant organs, plant tissues, plant cells, fungi, or bacteria, etc.

### BACKGROUND OF THE INVENTION

Nucleic acid extraction is a fundamental technology in the life field including agriculture, forestry, fishery, medicine, etc. Nucleic acids extracted from animal, plant, and microbial cells are used for various tests, such as RNA extraction from the human nasal patch, and cells in the throat, for the detection of neo-coronavirus and confirming the diagnosis of patients with neo-coronavirus infection. Our previously granted patents (U.S. Pat. No. 9,382,576, Japanese Patent License No. 5824747, Chinese Patent ZL 2011101292535, and PCT/CN2012/071598)) have mentioned the deficiencies of various generic nucleic acid extraction methods, especially RNA extraction methods, and we will not repeat them here.

Our previous patent (Chinese patent ZL 2011101292535) also has some shortcomings: 1. often requires heating; 2. can only extract RNA, not DNA; 3. cannot extract the RNA of vine and wood; 4. cannot extract DNA and RNA from blood. To overcome these problems, we have improved the original nucleic acid extraction method for life field applications.

### SUMMARY OF THE INVENTION

The purpose of the present invention is to provide a method for isolating and purifying Solid Nucleic Acids from biomaterials to overcome the shortcomings of existing technologies. The technical solution of the present invention is outlined as follows:

A method for isolating and purifying solid nucleic acids from biomaterials, comprising the following steps:

Step (1) Mix liquid containing naked nucleic acid with a 3.64M-5M alkali metal salt aqueous solution that acts as a precipitant at a volume ratio of 1:(1-11); centrifuge at room temperature and pour the supernatant into a new centrifuge tube;

Step (2) Proceed in one of the following three ways:

### Way I.

Add isopropanol to the new centrifuge tube containing the supernatant obtained in step (1) at a volume ratio of (1-4.4):1, mix well, allow it to stand at room temperature for 1-30 minutes, centrifuge, generate a white precipitate, discard all except the white precipitate, wash and obtain solid RNA or a mixture of solid RNA and solid DNA for preservation;

### Way II.

Add isopropanol to the new centrifuge tube containing the supernatant obtained in step (1) at a volume ratio of (1-4.4):1, mix well, and allow it to stand at room temperature for 1-30 minutes, add distilled water equivalent to (0.0714-0.1348) times the volume of the supernatant, mix well, centrifuge, generate a white precipitate, pour the liquid other than the white precipitate named the retention liquid into another new centrifuge tube, wash the white precipitate, and obtain solid DNA, or a mixture of solid RNA and solid DNA for preservation;

### Way III.

1) Add isopropanol to the new centrifuge tube containing the supernatant obtained in step (1) at a volume ratio of (1.5-1.76):1, mix well, allow it to stand at room temperature for 1-30 minutes, centrifuge, generate a white precipitate, discard the liquid named the retained liquid except for the white precipitate, washing the white precipitate, and obtaining solid RNA or a mixture of solid RNA and solid DNA for preservation;

2) Add distilled water to the new centrifuge tube containing the retained liquid, mix well, centrifuge at room temperature for 1-30 minutes, generate a white precipitate, discard all except the white precipitate, wash, and obtain solid DNA for preservation. The ratio of supernatant to distilled water is 1:(0.125-0.1705).

Said liquid containing naked nucleic acid is prepared by the following method:

homogenizing 16.27-162.8 mg of biomaterial with 1 ml of homogenization-dissociation reagent in proportion, to obtain a liquid containing naked nucleic acid;

Said homogenization-dissociation reagent is composed of formamide, an aqueous solution of alkali metal salts with a concentration of 5M-14M, and compounds for removal of secondary cellular metabolites in the ratio of 200 ml: 10-50 ml: 0-10 g.

Said biomaterial is animal organs, animal tissues, animal cells, plant organs, plant tissues, plant cells, fungi, or bacteria.

Said homogenization-dissociation reagent is composed of formamide in the ratio of 200 ml:10-50 ml:2-5 g, an aqueous solution of alkali metal salts in the concentration of 5 M-14 M, and a compound for removing cellular secondary metabolites.

Said alkali metal salt is lithium chloride or sodium chloride.

The washing is conducted with an aqueous solution of ethanol at a volume concentration of 70%-90%, centrifuged at 2000-16000 g for 10-60 s at RT, and the washing solution is poured off; then washed with absolute ethanol and air-dried.

Said compound for removal of cellular secondary metabolites is at least one of casein, polyvinylpyrrolidone 40, and cetyltrimethylammonium bromide.

The advantages of the present invention are as follows:

(1) The present invention uses a homogenization-dissociation reagent for homogenizing biological materials, which can be used to obtain a liquid containing naked nucleic acids simply without the need for heating, for the extraction of solid RNA and DNA.

(2) By adding compounds that remove secondary metabolites from cells to the homogenization-dissociation reagent, solid RNA and solid DNA can be extracted from vines, trees, and human blood, respectively.

(3) The four-phase RNA and DNA purification methods of the present invention (i.e. RNA solids precipitate at the bottom of the centrifuge tube while DNA and other impurities are located between the isopropyl alcohol upper phase and the high-salt lower phase, and DNA solids precipitate at the bottom of the centrifuge tube while other impurities are located between the isopropyl alcohol upper phase and the high-salt lower phase) can be used to obtain solid RNA and DNA completely free of protein and other impurities. The solid nucleic acids obtained (especially solid RNA) can be stored for up to one month at room temperature or up to one year at temperatures below −20° C.

### BRIEF DESCRIPTION OF DRAWINGS

FIG. 1 shows the 1×TAE, 1.0% agarose gel electrophoresis pattern of extracted young cabbage leaves nucleic acid sample, with a 500 bp DNA ladder as the DNA marker.

FIG. 2 shows the relationship between the volume and yield of nucleic acid in the liquid containing naked nucleic acids from young cabbage leaves.

FIG. 3 shows the relationship between the volume and recovery rate of nucleic acid in the liquid containing naked nucleic acids from young cabbage leaves.

FIG. 4 shows the relationship between the mass of young rose leaves in the liquid containing naked nucleic acids and the RNA yield.

FIG. 5 shows the relationship between the mass of young rose leaves in the liquid containing naked nucleic acids and the RNA recovery rate.

FIG. 6 shows the 1×TAE, 0.6% agarose gel electrophoresis pattern of the extracted solid nucleic acid from human finger blood (Example 3), with λ/Hind III DNA as the DNA marker.

FIG. 7 shows the 1×TAE, 0.6% agarose gel electrophoresis pattern of the extracted nucleic acid sample from EDTA anticoagulated venous blood of humans (Example 4), with λ/Hind III DNA as the DNA marker.

FIG. 8 shows the 1×TAE, 1.0% agarose gel electrophoresis pattern of the extracted nucleic acid sample E from mouse liver (Example 5) and the further separated nucleic acid samples F0 and F1 (Example 6), with 500 bp DNA ladder as the DNA marker.

FIG. 9 shows the 1×TAE, 1.0% agarose gel electrophoresis pattern of the extracted nucleic acid sample G0 from the mouse liver and the further separated nucleic acid samples G1 and G2 (Example 7), with 500 bp DNA ladder as the DNA marker.

FIG. 10 shows the 1×TAE, 1.0% agarose gel electrophoresis pattern of the isolated nucleic acid sample H from grape seedlings (Example 9), with DNA Marker III as the DNA marker.

FIG. 11 shows the 1×TAE, 1.0% agarose gel electrophoresis pattern of the isolated nucleic acid sample I from young grape leaves (Example 10), with DNA Marker III as the DNA marker.

FIG. 12 shows the 1×TAE, 1.0% agarose gel electrophoresis pattern of RNA samples J and K from carrot tuber (Examples 11 and 12), with a 500 bp DNA ladder as the DNA marker.

FIG. 13 shows the 1×TAE, 1.0% agarose gel electrophoresis pattern of the isolated nucleic acid sample L0 from rosebud petals (Example 13), with a 500 bp DNA ladder as the DNA marker.

FIG. 14 shows the 1×TAE, 1.0% agarose gel electrophoresis pattern of the isolated nucleic acid sample L1 from rosebud petals (Example 13), with Trans 2K Plus DNA as the DNA marker.

FIG. 15 shows the RIN measurement of the total RNA product from the isolated nucleic acid sample L1 from rosebud petals (Example 13), generated by Agilent 2100 Bioanalyzer.

### DETAILED DESCRIPTION OF THE INVENTION

The following examples are provided to enable those skilled in the art to understand the present invention, but do not limit the scope of the invention in any way.

### Example 1

A method for isolating and purifying solid nucleic acids from biomaterials and the relationship between the volume of naked nucleic acid liquid containing young cabbage leaves and the yield and recovery rate of nucleic acid, comprising the following steps:

(1) 300 mg of cabbage leaves and 5 ml of a homogenization-dissociation reagent (comprising 200 ml of formamide and 50 ml of 5M NaCl solution) were placed in a Dounce homogenizer on ice and quickly homogenized to obtain naked nucleic acid liquid containing cabbage leaves.

According to Table 1 (including Table 1-1, Table 1-2), eleven 1.5 ml centrifuge tubes were labeled and different volumes of naked nucleic acid liquid and 700 μl of alkaline metal salt precipitation solution (hereafter referred to as precipitant) (3.57 M NaCl, 1.14 M KCl water solution) were added to each tube. The tubes were repeatedly inverted to mix the liquid. After centrifugation at 12,000 g for 5 min at RT (i.e., at room temperature), the supernatant was poured into another centrifuge tube, and the approximate volume of the supernatant in each tube is shown in Table 1.

TABLE 1-1
Operating parameters and UV spectrophotometry results of nucleic acid samples from young cabbage leaves

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol.* | Vol. | Vol. | Vol. | liquid | vol. | vol. | Isopropanol |
| No. | (mg) | (μl) | (mg/ml) | (μl) | (μl) | Vol. | (μl) | (μl) | vol. |
| A −1 | 300 | 5000 | 60 | 150 | 700 | 4.67 | 800 | 500 | 1.60 |
| A0 | 300 | 5000 | 60 | 175 | 700 | 4.00 | 820 | 500 | 1.64 |
| A1 | 300 | 5000 | 60 | 200 | 700 | 3.50 | 840 | 500 | 1.68 |
| A2 | 300 | 5000 | 60 | 225 | 700 | 3.11 | 860 | 500 | 1.72 |
| A3 | 300 | 5000 | 60 | 250 | 700 | 2.80 | 880 | 500 | 1.76 |
| A4 | 300 | 5000 | 60 | 275 | 700 | 2.55 | 900 | 500 | 1.80 |
| A5 | 300 | 5000 | 60 | 300 | 700 | 2.33 | 920 | 500 | 1.84 |
| A6 | 300 | 5000 | 60 | 325 | 700 | 2.15 | 940 | 500 | 1.88 |
| A7 | 300 | 5000 | 60 | 350 | 700 | 2.00 | 960 | 500 | 1.92 |
| A8 | 300 | 5000 | 60 | 400 | 700 | 1.75 | 980 | 500 | 1.96 |
| A9 | 300 | 5000 | 60 | 450 | 700 | 1.56 | 1000 | 500 | 2.00 |

*Homo - Diss: homo genization-dissociation reagent; vol. volume. The following tables have the same situation, so we will not repeat the explanation.

TABLE 1-2
Operating parameters and UV spectrophotometry results of nucleic
acid samples from young cabbage leaves (Continued)

| Nucleic | | | | | |
|---|---|---|---|---|---|
| acid | | | | | Recovery |
| sample | Nucleic acid | | | Yield_{∧} | yield |
| No. | Type | OD260/280 | OD260/230 | (μg) | (μg/mg) |
| A-1 | — | — | — | 0 | 0 |
| A0 | RNA | 2.04 | 2.53 | 8.02 | 0.81 |
| A1 | RNA | 2.06 | 2.51 | 35.6 | 3.14 |
| A2 | RNA | 2.01 | 2.51 | 55.6 | 4.37 |
| A3 | RNA | 2.07 | 2.47 | 62.1 | 4.39 |
| A4 | RNA | 2.11 | 2.42 | 61.22 | 3.93 |
| A5 | RNA | 2.08 | 2.47 | 65.2 | 3.84 |
| A6 | RNA & DNA | 2.01 | 2.47 | 81.28 | 4.42 |
| A7 | RNA & DNA | 2.12 | 2.24 | 92.58 | 4.67 |
| A8 | RNA & DNA | 2.09 | 2.29 | 114.84 | 5.07 |
| A9 | RNA & DNA | 2.01 | 2.17 | 152.64 | 5.99 |

_{∧}Calculate the nucleic acid concentration according to the fact that 1 absorbance at 260 nm of nucleic acid sample is equivalent to a nucleic acid concentration of 40 ng/ml.

(2) 500 μl of isopropanol was added to each supernatant, and the centrifuge tubes were inverted several times to mix the liquid. After standing at RT for 15 min, the tube was centrifuged at 12,000 g for 5 min, and the isopropanol upper phase, the high-salt lower phase, and the impurities between the two liquid phases were discarded, leaving a white solid at the bottom of the tube.

The white solid in the centrifuge tube was washed with 1 ml of 90% vol. ethanol. After centrifugation at 12,000 g for 30 sec at RT, the washing liquid was discarded. The washing was repeated with absolute ethanol and the solid was air dried.

No solid was found in tube A-1, which was discarded. On the ice, 175, 200, 225, 250, 275, 300, 325, 350, 400, and 450 μl of ice-cold high-purity water were added to centrifuge tubes AO to A9 to dissolve the nucleic acid solids inside and obtain nucleic acid samples.

UV spectrophotometry results: The nucleic acid solutions were measured using the Thermo-Fisher NanoDrop 2000 ultra-micro spectrophotometer. The results are shown in Table 1; all nucleic acid samples have an OD260/280 value close to 2.0, indicating that the extracted nucleic acid samples are high-purity RNA or RNA contaminated with DNA. OD260/230 values for all nucleic acid samples also indicate that the samples are not contaminated with salt or insoluble material.

Agarose gel electrophoresis: 2 μl of the above AO to A9 solutions were electrophoresed in a 1.0% agarose gel (1×TAE electrophoresis buffer, stained with ethidium bromide) at 4 V/cm for 30 minutes, with an ice bag placed over the electrophoresis tank to lower the temperature.

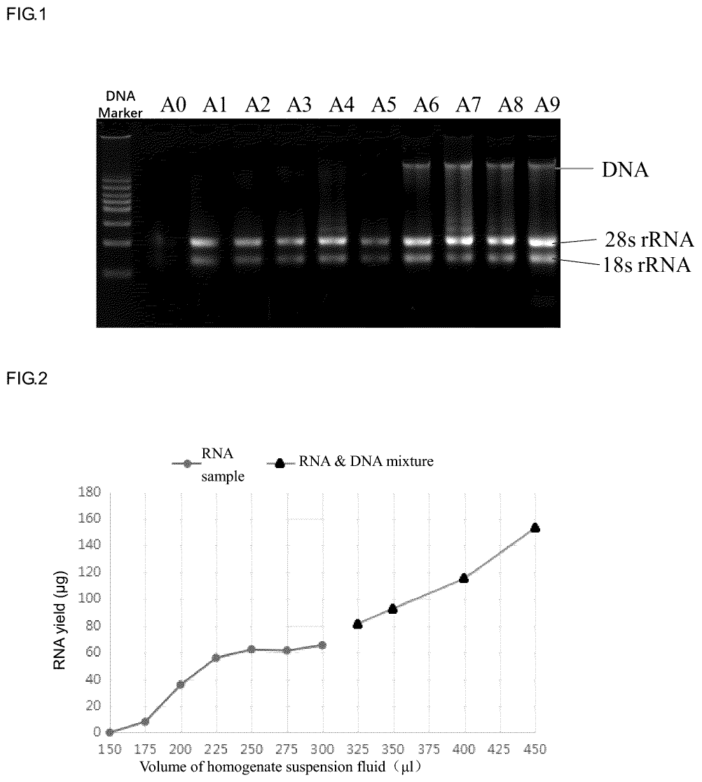

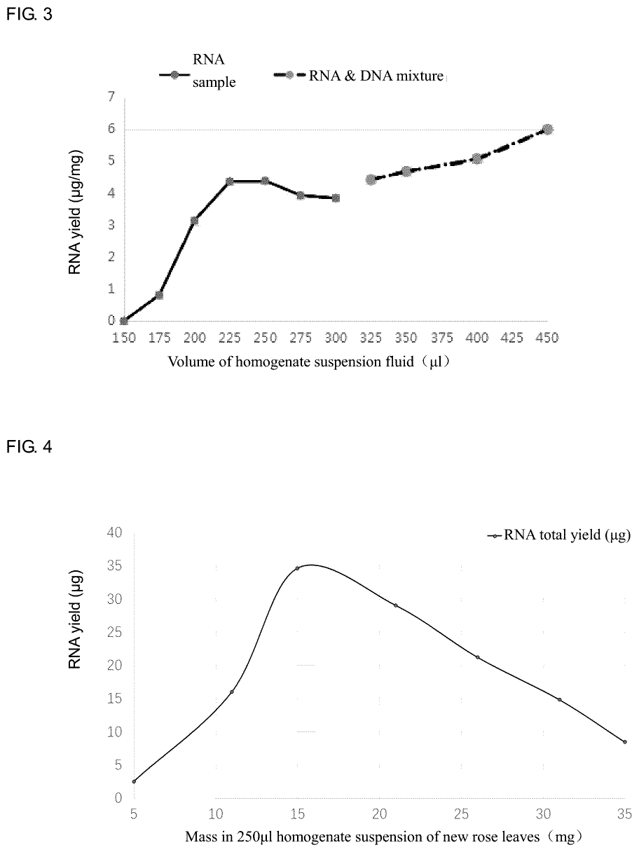

Agarose gel electrophoresis results: As shown in FIG. 1, the margins of the 18s rRNA and 28s rRNA of nucleic acid samples AO to A9 are clean, indicating that the RNA molecules inside are not degraded. Nucleic acid samples AO to A5 have no corresponding DNA bands, indicating that these nucleic acid samples contain only RNA components without DNA components. Nucleic acid samples A6-A9 contain higher molecular weight DNA components and also RNA components. Taken together with FIG. 2 and FIG. 3, it can be concluded that for the extraction of pure RNA, the highest yield and the greatest recovery rate is obtained by using 225 μl to 275 μl of naked nucleic acid liquid mixed with 700 μl of precipitant. For the extraction of solid nucleic acids containing both DNA and RNA, 400 μl of naked nucleic acid liquid mixed with 700 μl of the precipitant is best.

Conclusion: The results of Example 1 show that high-quality solid RNAs can be extracted from cabbage leaves using the method of the invention and that nucleic acid solids containing DNA and RNA can also be obtained. Solid RNAs with both the highest yield and the highest recovery rate can be obtained by using 250 μl of naked nucleic acid liquid and 700 μl of precipitant. The best result for the extraction of nucleic acid solids containing DNA and RNA is obtained by using 400 μl Expanded Nucleic Acid Liquid mixed with 700 μl the precipitant.

### Example 2

A method for isolating and purifying solid nucleic acids from biomaterials, and the relationship between the mass of young rose leaves in the naked nucleic acid liquid and the yield and recovery rate of RNA, comprising the following steps

(1)In each labeled centrifuge tube, young rose leaves were weighed according to Table 2, including Table 2-1, Table 2-2, and a homogenization-dissociation reagent (200 ml formamide and 50 ml 5M NaCl aqueous solution, 5 g casein (compound for removing cellular secondary metabolites) in a 500 ml reagent bottle sterilized in an autoclave at 121° ° C. for 20 min), so that the sum of the milligrams of young rose leaves and the microliters of homogenization-dissociation reagent were about 250. Then 2 mg of 0.5 mm diameter ceramic beads and two 2 mm diameter ceramic beads were added to each centrifuge tube and homogenized for 1 min at 50 Hz to obtain approximately 250 μl of naked nucleic acid liquid. Then 700 μl of precipitant (same as the precipitant in Example 1) was added, the centrifuge tube was inverted repeatedly to mix the liquid, and after centrifugation at 12,000 g for 5 min at RT, the supernatant was transferred to another centrifuge tube.

TABLE 2-1
Operating parameters and UV spectrophotometry results of nucleic acid samples from young rose leaves

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| B0 | 4 | 246 | 16.26 | 250 | 700 | 2.8 | 880 | 500 | 1.76 |
| B1 | 11 | 239 | 46.03 | 250 | 700 | 2.8 | 880 | 500 | 1.76 |
| B2 | 15 | 235 | 63.83 | 250 | 700 | 2.8 | 880 | 500 | 1.76 |
| B3 | 21 | 229 | 91.7 | 250 | 700 | 2.8 | 880 | 500 | 1.76 |
| B4 | 26 | 224 | 116.07 | 250 | 700 | 2.8 | 880 | 500 | 1.76 |
| B5 | 31 | 219 | 141.55 | 250 | 700 | 2.8 | 880 | 500 | 1.76 |
| B6 | 35 | 215 | 162.79 | 250 | 700 | 2.8 | 880 | 500 | 1.76 |

TABLE 2-2
Operating parameters and UV spectrophotometry results of nucleic
acid samples from young rose leaves (Continued)

| Nucleic | | | | | Recovery |
|---|---|---|---|---|---|
| acid | Nucleic acid | | | Yield | yield |
| sample No. | Type | OD260/280 | OD260/230 | (μg) | (μg/mg) |
| B0 | RNA | 2.18 | 2.43 | 2.5 | 0.63 |
| B1 | RNA | 2.13 | 2.48 | 16 | 1.45 |
| B2 | RNA | 2.15 | 2.22 | 34.57 | 2.3 |
| B3 | RNA | 2.17 | 2.26 | 29 | 1.38 |
| B4 | RNA | 2.14 | 2.5 | 21.18 | 0.81 |
| B5 | RNA | 2.12 | 2.46 | 14.8 | 0.48 |
| B6 | RNA | 2.12 | 2.42 | 8.42 | 0.24 |

(2) Same as step (2) in Example 1.

To each centrifuge tube on ice, 100 μl of ice-bathed high-purity water was added to dissolve the white solids inside, and nucleic acid samples B0 to B6 were obtained sequentially.

Results of UV spectrophotometry: The assay was carried out as in Example 1. The results are shown in Table 2, FIG. 4, and FIG. 5: Samples B1 to B3 gave better yield and rate of RNA recovery, with the highest yield and rate of RNA recovery obtained using 15 mg of young rose leaves and 235 μl of homogenization-dissociation reagent to prepare the nucleic acid-containing liquid.

Conclusion: Using the nucleic acid-containing liquid prepared with 15 mg young rose leaves and 235 μl homogenization-dissociation reagent (such as nucleic acid sample B2 in Table 2) for RNA extraction can simultaneously achieve the best both RNA yield and recovery rate.

### Example 3

A method for isolating and purifying solid nucleic acid from biomaterials (human blood), comprising the following steps:

(1) At room temperature, 40 μl (about 40 mg) of the finger blood of the inventor Yang Xianglong was added to 250 μl of homogenization-dissociation reagent (200 ml of formamide and 50 ml of 5M NaCl water solution, 2 g of casein) in a 1.5 ml centrifuge tube, and about 20 mg of 1.0 mm diameter ceramic beads were added. Homogenization was carried out on a ball mill for 1 min at a frequency of 50, yielding a liquid containing 290 μl of naked nucleic acid.

After homogenization, 290 μl of the naked nucleic acid-containing liquid was taken, 700 μl of a precipitant (same as in Example 1) was added, the liquid was vortexed in the centrifuge tube, centrifuged at 12,000 g for 5 min at RT, and the supernatant was poured into another centrifuge tube; the volume of the supernatant was approximately 920 μl (see Table 3, including Tables 3-1 and 3-2).

TABLE 3-1
Operating parameters and UV spectrophotometry results of nucleic acid samples from human finger blood

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| C | 40 | 250 | 160 | 290 | 700 | 2.41 | 920 | 500 | 1.84 |

Note:
*The nucleic acid sample herein contains both DNA and RNA, and the OD260 value cannot be used to accurately determine the nucleic acid content yield.

TABLE 3-2
Operating parameters and UV spectrophotometry results of
nucleic acid samples from human finger blood (continued)

| | | Vol. of | | | | |
|---|---|---|---|---|---|---|
| | | water | | | | |
| | | added/ | | | | |
| Nucleic | Vol. of | Super- | | | | |
| acid | water | natant | Nucleic | | | |
| sample | added | vol. | acid | OD260/ | OD260/ | Yield* |
| No. | (μl) | (μl) | type | 280 | 230 | (g) |
| C | 65.7 | 0.0714 | DNA&RNA | 1.94 | 2.35 | — |

Note:
*The nucleic acid sample herein contains both DNA and RNA, and the OD260 value cannot be used to accurately determine the nucleic acid content yield.

(2) 500 μl of isopropanol was added to the supernatant, stirred well, and allowed to stand for 1 min at RT. Add 65.7 μl of distilled water to the centrifuge tube, mix well, and centrifuge at 12,000 g for 5 minutes. Discarded the upper and lower phase liquids and the impurities between the two phases, then a white solid at the bottom of the original centrifuge tube was obtained.

Washing the white solid as in Example 1.

On the ice, 25 μl of ultrapure water is added to dissolve the white solid in the centrifuge tube, giving nucleic acid sample C, which is stored at −20° C. or detected.

UV spectrophotometry results: The assay was performed as in Example 1. The results are given in Table 3.

Agarose gel electrophoresis: as in Example 1, except that 0.6% agarose concentration was used.

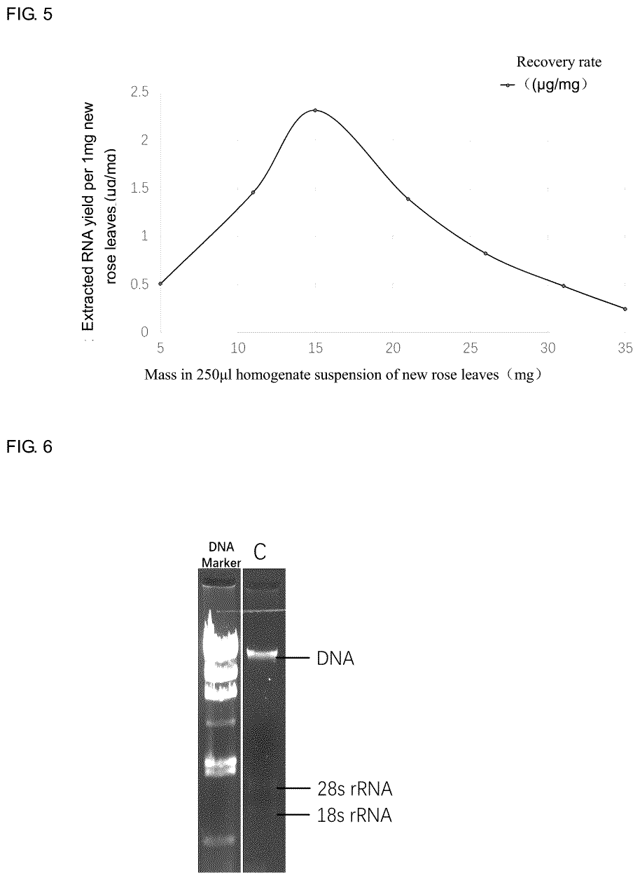

Agarose gel electrophoresis result: The electrophoresis profile is shown in FIG. 6, which shows that there was a DNA band of approximately 23 kb in size and 28s rRNA and 18s rRNA bands in nucleic acid sample C, which proves that the extracted nucleic acid sample C contains intact RNA and DNA molecules, and the RNA molecule is not degraded.

Conclusion: The results of Example 3 illustrate that solid nucleic acids containing both intact RNA and DNA can be obtained from human finger blood using the method of the present invention.

### Example 4

A method for isolating and purifying solid nucleic acid from biomaterials (human EDTA anticoagulated venous blood) comprising the steps of

(1) 0.4 ml of healthy human EDTA anticoagulated venous blood discarded from a health check center in Tianjin was taken and placed in a 1.5 ml centrifuge tube. 1 ml of distilled water was added, and the centrifuge tube was inverted several times and mixed to lyse the red blood cells. Centrifugated for 1 min at 12,000 g at RT and discard the liquid. 0.5 ml of distilled water was added, the centrifuge tube was inverted several times to suspend the precipitate at the bottom of the tube, centrifuged again, and the liquid was discarded. After the third centrifugation, a microsample was used to remove the remaining liquid to obtain approximately 15 mg (equivalent to 15 μl) of nucleated cells in human blood. See Table 4 (including Table 4-1, Table 4-2) for the precipitate obtained.

TABLE 4-1
Operating parameters and UV spectrophotometry results of nucleic
acid samples from human EDTA anticoagulated venous blood

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| D | ~15 | 250 | 60 | 265 | 700 | 2.64 | 890 | 500 | 1.78 |

TABLE 4-2
Operating parameters and UV spectrophotometry
results of nucleic acid samples from human
EDTA anticoagulated venous blood (Continued)

| | | Vol. of | | | | |
|---|---|---|---|---|---|---|
| | | water | | | | |
| Nucleic | Vol. of | added/ | | | | |
| acid | water | Supermatant | Nucleic | | | |
| sample | added | vol. | acid | OD260/ | OD260/ | Yield |
| No. | (μl) | (μl) | Type | 280 | 230 | (μg) |
| D | 120 | 0.1348 | DNA | 1.82 | 1.77 | 8.91 |

Suspended the precipitate in the centrifuge tube by adding 250 μl of homogenization-dissociation reagent (the same as in Example 2) and inverting the tube several times until the suspension changes from cloudy to clear, giving a liquid containing naked DNA.

700 μl of precipitant (the same as in Example 1) was added to the centrifuge tube, the liquid in the tube was vortexed, centrifuged at 12,000 g for 5 min at RT and the supernatant was poured into another centrifuge tube. The volume of the supernatant is approximately 900 μl.

(2) 500 μl of isopropanol was added to the supernatant, mixed, and left at RT for 30 min. 120 μl of distilled water was added to the centrifuge tube, mixed, and centrifuged at 12,000 g for 5 min at RT, and the top and bottom liquid and solid impurities between the two liquid phases were discarded, leaving a white solid at the bottom of the original centrifuge tube.

The white solid was washed as in Example 1.

On the ice, 100 μl of ice-bath high-purity water was added to dissolve the solid in the centrifuge tube to obtain nucleic acid sample D, which was stored at −20° C. or detected.

UV spectrophotometry results: The assay was carried out as in Example 1 and the results are shown in Table 4 that OD260/280 is 1.82. This indicates that the DNA sample obtained is of high purity, with no protein or RNA contamination.

Agarose gel electrophoresis: same as Example 1 except that 0.6% agarose concentration was used.

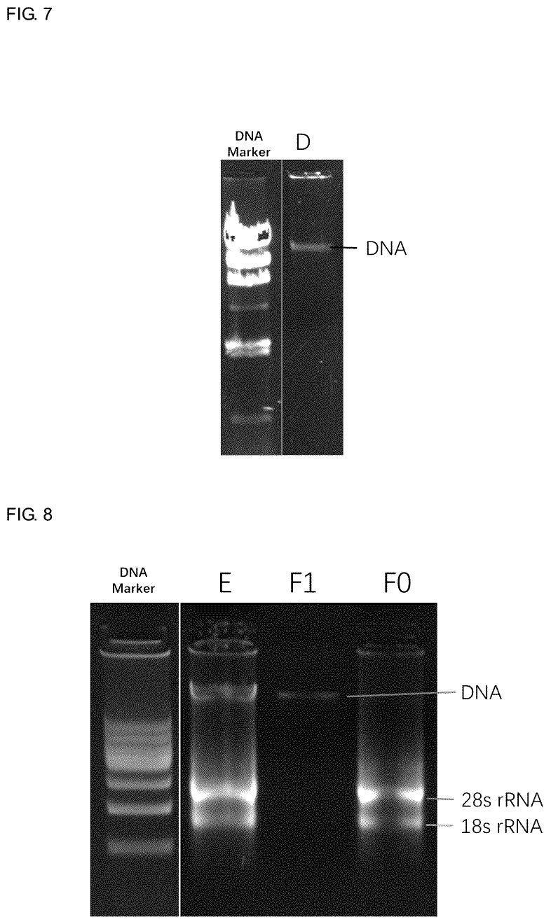

Agarose gel electrophoresis result: As shown in FIG. 7, a DNA band of approximately 23 kb was observed in nucleic acid sample D, indicating that the extracted nucleic acid sample D is human blood DNA molecules and has not been degraded. No RNA band was observed.

Conclusion: The results of Example 4 demonstrate that the method of the present invention can be used to obtain high-quality solid DNA from human EDTA anticoagulated blood and that the solid DNA is free of RNA contamination.

### Example 5

A method for isolating and purifying solid nucleic acid from biomaterials (mouse liver), comprising the following steps:

(1) 500 mg of mouse liver and 8 ml of homogenization-dissociation reagent (as in Example 1) were placed in a 10 ml Dounce homogenizer on ice and rapidly homogenized to obtain a liquid containing naked nucleic acids.

7 ml of the liquid was placed in a 50 ml centrifuge tube, 14 ml of precipitant (same as in Example 1) was added and the centrifuge tube was inverted several times to mix the liquid inside. The centrifuge tube was centrifuged at 2,000 g for 30 min at RT; then the supernatant in the centrifuge tube was transferred to another 50 ml centrifuge tube; the volume of the supernatant was about 19 ml, as shown in Table 5 (including Table 5-1, Table 5-2).

(2) 10 ml of isopropanol was added to the supernatant, mixed, and allowed to stand at RT for 30 min; the centrifuge tube was centrifuged at 2000 g for 30 min at RT; then the liquid in the centrifuge tube was poured off, and a white solid was visible at the bottom of the centrifuge tube.

Washing of the white solid was the same as in Example 1, with two differences: 1) the washing was performed with 20 ml of 90% ethanol and 20 ml of absolute ethanol washing solutions, respectively; 2) the centrifugation was performed at 2,000 g for 2 min at RT.

On the ice, 6 ml of ice-bathed ultrapure water was added to dissolve the solids in the centrifuge tube, yielding mouse liver nucleic acid sample E for storage at −20° C. or detection.

UV spectrophotometry results: The assay was performed as in Example 1 and the results are shown in Table 5.

Agarose gel electrophoresis test: As in Example 1.

TABLE 5-1
Operating parameters and UV spectrophotometry results of nucleic acid samples from mouse liver (1)

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| E | 500 | 8000 | 62.5 | 7000 | 14000 | 2.00 | 19000 | 10000 | 1.90 |
| F0 | 50 | 150 \hat{ } | — | 200 | 700 | 3.50 | 880 | 500 | 1.76 |

| | |
|---|---|
| F1 | Retention fluid from F0 |

Note:
*The homogenization-dissociation reagent herein is used as a dilution solution for the nucleic acid solution, similar to the dilution of nucleic acid solution with distilled water in Example 7
\hat{ } The nucleic acid sample herein contains DNA and RNA, and the OD260 value cannot be used to accurately determine the yield

TABLE 5-2
Operating parameters and UV spectrophotometry results of
nucleic acid samples from mouse liver (1) (Continued)

| | | Vol. of | | | | |
|---|---|---|---|---|---|---|
| | | water | | | | |
| Nucleic | Vol. of | added/ | | | | |
| acid | water | Supermatant | Nucleic | | | |
| sample | added | vol. | acid | OD260/ | OD260/ | Yield |
| No. | (μl) | (μl) | Type | 280 | 230 | (μg) |
| E | None | None | RNA&DNA | 1.97 | 2.58 | — * |
| F0 | None | None | RNA | 2.08 | 2.26 | 15.06 |
| F1 | 150 | 0.1705 | DNA | 1.89 | 2.34 | 11.99 |

Agarose gel electrophoresis test: same as in Example 1.

Agarose gel electrophoresis result: The electrophoresis pattern is shown in FIG. 8: 1) The 18s rRNA and 28s rRNA bands of nucleic acid sample E were neatly arranged at the edge, indicating that the extracted nucleic acid sample E contains complete RNA molecules; 2) The nucleic acid sample E contained about 23 kb DNA bands, indicating that the method of the present invention can be used to extract DNA and intact RNA molecules simultaneously.

Conclusion: The results of Example 5 demonstrate that the method of the present invention can be used to obtain nucleic acid solids containing both DNA and complete RNA molecules from mouse liver.

### Example 6 Method for Separating and Purifying Nucleic Acid Components from a mixed solution of RNA and DNA (1)

A method for isolating and purifying solid nucleic acid from biomaterials, comprising the following steps:

(1) Mixed 50 μl of nucleic acid sample E from Example 5 and 150 μl of a dissociation reagent (the same as the homogenization-dissociation reagent in Example 1 but used as a diluent for the nucleic acid solution) at RT to obtain a solution containing naked nucleic acids.

Then 700 μl of precipitant (same as the precipitant in Example 1) was added, the centrifuge tube was inverted several times to mix the liquid, centrifuged at 12,000 g for 5 min at RT, and 880 μl of the supernatant was transferred to another centrifuge tube. (See Table 5 for details)

(2)500 μl of isopropanol was added to the supernatant, mixed well, and allowed to stand at RT for 30 min. After centrifugation at 12,000 g for 5 min at RT, the liquid was poured into a new centrifuge tube, leaving a white solid at the bottom of the original centrifuge tube.

(3) 150 μl of distilled water was added to the new centrifuge tube containing the retention liquid, mixed well, and centrifuged at 12,000 g for 5 min at RT, a white precipitate was obtained, and the other materials except the white precipitate were discarded and washed. Solid DNA was obtained and stored. The ratio of the supernatant to distilled water was 1:0.1705.

To the centrifuge tube containing the white solids obtained in steps (2) and (3), 1 ml of 70% ethanol-water solution was added to wash them; after centrifugation at 12,000 g at RT for 30 sec, the washing solution was discarded. Repeat washing with absolute ethanol. The solid was dried.

50 μl of ice-bath ultrapure water was added to dissolve the white solid in the centrifuge tube on ice, nucleic acid samples F0 and F1 were obtained, stored in a refrigerator at −20° C., or detected.

UV spectrophotometry results: The assay was performed as in Example 1 and the results are shown in Table 5.

Agarose gel electrophoresis: as in Example 1.

Agarose gel electrophoresis results: The electrophoretogram shown in FIG. 8 indicates that 1) nucleic acid sample F0 contains neatly arranged 18s rRNA and 28s rRNA bands at the edge, proving that the purified white solid is an RNA sample and has not been degraded; 2) nucleic acid sample F1 contains a DNA band spectrum of approximately 23 kb, proving that the extracted white solid is a DNA sample. This indicates that the method of the present invention can separate the DNA component and the intact RNA component from nucleic acid samples.

Conclusion: The results of Example 6 show that the method of the present invention can easily separate the DNA and RNA components from the whole nucleic acid sample and does not damage the integrity of the RNA molecule.

### Example 7 Method for separating and purifying nucleic acid components from a mixed solution of RNA and DNA (2)

A method for isolating and purifying solid nucleic acid from biomaterials, comprising the following steps:

(1) At room temperature, 50 μl of mouse liver whole nucleic acid sample (containing DNA and RNA solutions, nucleic acid sample G0), obtained by the same method as in Example 5 and stored at −20° C. for one year, was mixed with 150 μl of ice-bathed high purity water in a 1.5 ml centrifuge tube to obtain a solution containing naked nucleic acids.

400 μl of precipitant (5M NaCl water solution) was added to the centrifuge tube and inverted several times to mix the liquid in the centrifuge tube (see Table 6, including Table 6-1, Table 6-2).

TABLE 6-1
Operating parameters and UV spectrophotometry results of nucleic acid samples from mouse liver (2)

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol | Isopropanol |
| No. | (mg) | (μl) | (mg/ml) | (μl) | (μl) | Vol. | (μl) | (μl) | vol. |
| G0 | Reference the nucleic acid | | | | | | | | |
| | sample E corresponding data | | | | | | | | |
| G1 | 50 | 150 | — | 200 | 400 | 2 | 600 | 400 | 1.5 |
| G2 | Retention fluid from G1 | | | | | | | | |
| G3 | 50 | 0 | — | 50 | 550 | 11 | 600 | 400 | 1.5 |

| | |
|---|---|
| G4 | Retention fluid from G3 |

Note:
*The nucleic acid sample herein contains DNA and RNA, and the OD260 value cannot be used to accurately determine the yield

TABLE 6-2
Operating parameters and UV spectrophotometry results
of nucleic acid samples from mouse liver (2)

| | | Vol. of | | | | |
|---|---|---|---|---|---|---|
| | | water | | | | |
| Nucleic | Vol. of | added/ | | | | |
| acid | water | Supermatant | Nucleic | | | |
| sample | added | vol. | acid | OD260/ | OD260/ | Yield |
| No. | (μl) | (μl) | Type | 280 | 230 | (μg) |
| G0 | None | None | RNA&DNA | 1.99 | 2.11 | — * |
| G1 | None | None | RNA | 2.06 | 2.33 | 4.73 |
| G2 | 75 | 0.125 | DNA | 1.84 | 2.39 | 3.9 |
| G3 | None | None | RNA | 2.04 | 2.3 | 4.64 |
| G4 | 75 | 0.125 | DNA | 1.83 | 2.34 | 3.8 |

Note:
* The nucleic acid sample herein contains DNA and RNA, and the OD260 value cannot be used to accurately determine the yield

(2) Add 400 μl of isopropanol to the centrifuge tube and mix well. Allow standing at RT for 1 minute. Centrifuge at 12,000 g for 5 minutes at RT. Transfer the liquid into a new 1.5 ml centrifuge tube, leaving the white solid at the bottom of the original centrifuge tube.

(3) Add 75 μl of ultrapure water to the new centrifuge tube from step (2) and invert the centrifuge tube several times to mix the liquid completely. The tube was centrifuged at 12,000 g for 5 minutes at RT to obtain a white precipitate. Everything except the white precipitate was discarded and washed, and the Solid DNA was obtained and stored. The ratio of supernatant to distilled water is 1:0.125.

Wash the white solid obtained in steps (2) and (3) as described in Example 6. Dissolve the white solid in the centrifuge tube obtained in steps (2) and (3) by adding 50 μl of ice-bath high-purity water on the ice to give nucleic acid samples G1 and G2. These were stored or assayed in a refrigerator at −20° C.

UV spectrophotometry results: The assay was carried out as in Example 1 and the results are given in Table 6.

Agarose gel electrophoresis: As in Example 1.

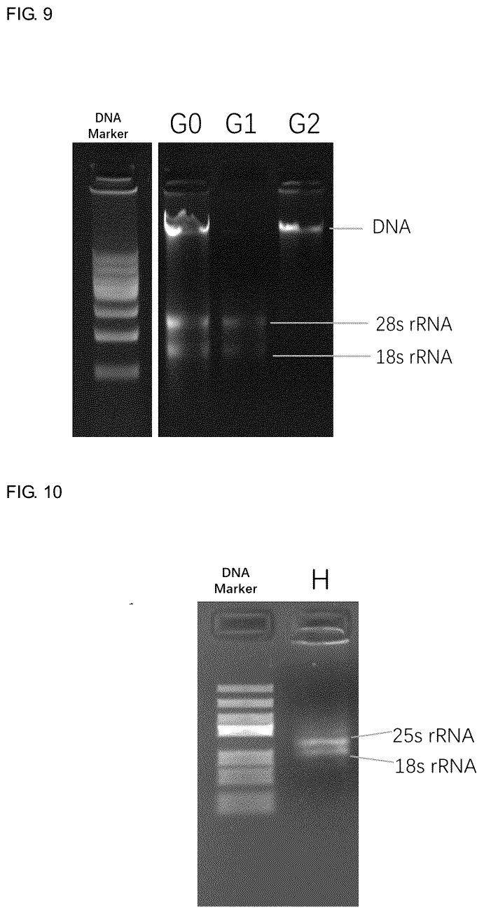

Result of agarose gel electrophoresis: The electrophoresis map is shown in FIG. 9, which shows that 1) nucleic acid sample G0 contained complete 18s rRNA, 28s rRNA, and about 23 kb DNA band spectrum, which shows that the DNA and complete RNA molecules extracted by the method of the invention can be stored at −20ºC for one year without decomposition; 2) nucleic acid sample G1 contains clean 18s rRNA, 28s rRNA bands at the edge, indicating that the RNA molecules in nucleic acid sample G0 were not degraded during the extraction process; 3) nucleic acid sample G2 contains approximately 23 kb DNA band spectrum. The complete electrophoresis results indicate that the invention method can be used to separate the DNA component and the complete RNA component in the nucleic acid sample at the same time.

Conclusion: The results of Example 7 show that the method of the present invention can easily separate DNA and RNA molecules in whole nucleic acid samples. At the same time, it is shown that the RNA component in the nucleic acid solution obtained by the method of Example 5 can be stored in a refrigerator at −20° C. for one year without decomposition.

### Example 8 Method for separating and purifying nucleic acid components from a mixed solution of RNA and DNA (3)

A method for isolating and purifying solid nucleic acid from biomaterials, comprising the following steps:

(1) 50 μl of mouse liver whole nucleic acid DNA and RNA solution (nucleic acid sample G0) from the same method as in Example 5 and stored at −20° ° C. for one year was added to a 1.5 ml centrifuge tube at RT to obtain a solution containing naked nucleic acid.

Added 550 μl of precipitant (3.636 M NaCl aqueous solution) to the centrifuge tube and inverted several times to mix the liquid (see Table 6).

(2) Same as step (2) in Example 7.

(3) Same as step (3) in Example 7.

Washed the white solids obtained in steps (2) and (3) as in Example 7. To dissolve the white solids in the centrifuge tube from steps (2) and (3), 50 μl of ice-bath high-purity water was added separately on ice to give nucleic acid samples G3 and G4, which were stored in a refrigerator at −20° C. or detected.

UV spectrophotometry results: The assay was performed as in Example 1. The results for nucleic acid samples G3 and G4 are given in Table 6.

Agarose gel electrophoresis: as in Example 1,

Result of agarose gel electrophoresis: nucleic acid samples G3 and G4 were like nucleic acid samples G1 and G2 in Example 6 and are omitted (see the electrophoretic spectrum in FIG. 8).

Conclusion: same as in Example 7.

### Example 9

Extraction of solid RNA from young grape leaves (1)

A method for isolating and purifying solid nucleic acids from biomaterials, comprising the following steps:

(1) 60 mg of young grape leaves and 1000 μl of homogenization-dissociation reagent (200 ml of formamide, 50 ml of 5M NaCl aqueous solution, 5 g of polyvinylpyrrolidone 40 (PVP40) and 5 g of cetyltrimethylammonium bromide (CTAB) in a flask, dissolving solids in a water bath at)90° ° C. were added to a 1 ml Dounce homogenizer at RT and the mixture was rapidly and thoroughly homogenized. The homogenate was then transferred to a 1.5 ml centrifuge tube and centrifuged at 1200 g for 1 min at RT to obtain a supernatant containing 250 μl of naked RNA in a new 1.5 ml centrifuge tube.

Polyvinylpyrrolidone 40 and cetyltrimethylammonium bromide are compounds used to remove secondary cellular metabolites.

Added 700 μl of precipitant (same as the precipitant in Example 1), inverted the centrifuge tube several times to mix the liquid, and centrifuged at 12,000 g for 5 min at RT. The supernatant was transferred to another centrifuge tube. The volume of the supernatant is 880 μl, as shown in Table 7 (including Table 7-1 and Table 7-2).

(2) 200 μl of isopropanol was added to the supernatant, and the centrifuge tube was inverted several times to mix the liquid and allowed to stand at RT for 1 minute. After centrifugation at 12,000 g for 5 minutes at RT, the isopropanol phase, the high salt phase, and the solid phase of the impurities between the two liquid phases were poured out to obtain a white solid at the bottom of the centrifuge tube.

TABLE 7-1
Operating parameters and UV spectrophotometry results of nucleic acid samples from young grape leaves

| | | | Biomaterial | | | Precipitant | | | |
|---|---|---|---|---|---|---|---|---|---|
| | | | mass/ | Naked | | Vol./ | | | |
| | | | Homo- | nucleic | | Naked | | | |
| Nucleic | | Homo- | Diss | acid | | nucleic | | | Supermatant |
| acid | Biomaterial | Diss | Vol. | liquid | Precipitant | acid | Supermatant | Isopropanol | vol./ |
| sample | mass | Vol. | (mg/ | Vol. | Vol. | liquid | vol. | vol. | Isopropanol |
| No. | (mg) | (μl) | ml) | (μl) | (μl) | Vol. | (μl) | (μl) | vol. |
| H | 60 | 1000 | 60 | 250 | 700 | 2.8 | 880 | 200 | 4.4 |

TABLE 7-2
Operating parameters and UV spectrophotometry results of nucleic
acid samples from young grape leaves (continued)

| Nucleic acid | Nucleic acid | | | |
|---|---|---|---|---|
| sample No. | Type | OD260/280 | OD260/230 | Yield (μg) |
| H | RNA | 2.13 | 2.28 | 20.355 |

The washing of the white solid in step (2) is the same as in Example 6.

Added 200 μl of ice-bath ultrapure water on the ice to dissolve the solid obtained in step (3) to obtain the nucleic acid sample H, which was stored at −20° C. or detected.

UV spectrophotometry results: The assay was performed as in Example 1 and the results are shown in Table 7.

Agarose gel electrophoresis: as in Example 1.

Agarose gel electrophoresis results: the electrophoretic profile in FIG. 10 shows the presence of 18s rRNA and 25s rRNA in nucleic acid sample H, and the edges of the band are complete, indicating that the RNA molecules in the extracted nucleic acid sample H have not been degraded.

Conclusion: The results of Example 9 show that RNA molecules can be extracted from grape leaves using the method of the present invention.

### Example 10 Extraction of Solid RNA from Young Grape Leaves (2)

A method for isolating and purifying solid nucleic acids from biomaterials, comprising the following steps:

(1) 60 mg of young grape leaves were mixed with 1 mL of a homogenization-dissociation reagent (2 mL of formamide containing 40 mg of casein was placed in a glass test tube, heated in boiling water to form a homogeneous solution, cooled to room temperature, then mixed with 0.1 mL of 14 M LiCl) in a 1 ml Dounce homogenizer and thoroughly homogenized to obtain a liquid containing naked RNA.

250 μl of this liquid was transferred to a 1.50 mL centrifuge tube and mixed with 700 μl of precipitant (the same as in Example 1) by inversion of the tube several times. After centrifugation at 12,000 g for 5 min at RT, the supernatant was transferred to another centrifuge tube as indicated in Table 8 (including Tables 8-1, 8-2).

TABLE 8-1
Operating parameters and UV spectrophotometry results of nucleic acid samples from young grape leaves 2

| | | | Biomaterial | | | Precipitant | | | |
|---|---|---|---|---|---|---|---|---|---|
| | | | mass/ | Naked | | Vol./ | | | |
| | | | Homo- | nucleic | | Naked | | | |
| Nucleic | | Homo- | Diss | acid | | nucleic | | | Supermatant |
| acid | Biomaterial | Diss | Vol. | liquid | Precipitant | acid | Supermatant | Isopropanol | vol./ |
| sample | mass | Vol. | (mg/ | Vol. | Vol. | liquid | vol. | vol. | Isopropanol |
| No. | (mg) | (μl) | ml) | (μl) | (μl) | Vol. | (μl) | (μl) | vol. |
| I | 60 | 1000 | 60 | 250 | 700 | 2.8 | 880 | 500 | 1.76 |

TABLE 8-2
Operating parameters and UV spectrophotometry results of nucleic
acid samples from young grape leaves 2 (continued)

| Nucleic acid | Nucleic acid | | | |
|---|---|---|---|---|
| sample No. | Type | OD260/280 | OD260/230 | Yield (μg) |
| I | RNA | 2.1 | 2.16 | 28.1 |

(2) Same as step (2) of Example 9.

The washing of the white solid in step (2) was the same as in Example 9.

100 μl of ice-bath high-purity water was added to dissolve the solid nucleic acid in the centrifuge tube on ice, which became nucleic acid sample I. It was stored in a −20° C. freezer for one year or tested.

UV spectrophotometry results: The assay was performed as in Example 1 and the results are shown in Table 8.

Agarose gel electrophoresis: as in Example 1.

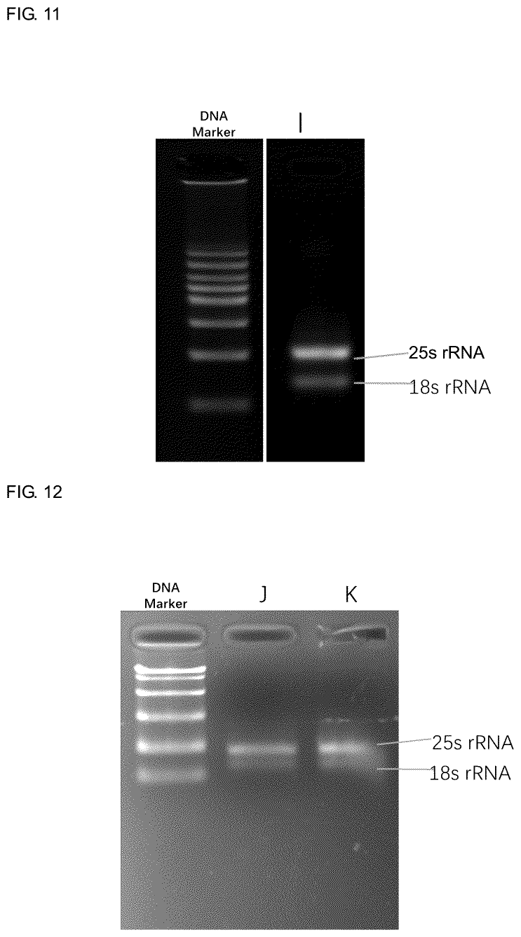

Agarose gel electrophoresis result: The electrophoretic pattern shown in FIG. 11 proved that the 18s rRNA and 25s rRNA in the sample I were completely intact, indicating that the extracted grape leaf RNA molecules were not degraded.

Conclusion: The results of Example 10 show that RNA in grape leaves can be extracted by the method of the present invention and that the solid RNA can be stored in an aqueous solution at −20° C. for one year without significant degradation.

### Example 11 Extraction of Solid RNA from Carrot Tuber

A method for isolating and purifying nucleic acid solids from biomaterial, comprising the following steps:

(1) 20 mg of carrot tuber tissue and 230 μl of homogenization-dissociation reagent (200 ml of formamide, 10 ml of 14M LiCI solution, and 4.2 g of polyvinylpyrrolidone 40 (PVP40) dissolved in a water bath at)90° ° C. were placed in a 1.5 ml centrifuge tube and four 2 mm diameter steel beads were added. The mixture was homogenized 4 times for 15 sec at 50 Hz to obtain a liquid containing naked nucleic acid.

700 μl of precipitant (the same as in Example 1) was added and the centrifuge tube was inverted several times to mix the liquid. After centrifugation at 12,000 g for 5 min at RT, the supernatant was transferred to another centrifuge tube as shown in Table 9 (including Table 9-1 and Table 9-2).

TABLE 9-1
Operating parameters and UV spectrophotometry results of nucleic acid samples from carrot tuber

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| J | 20 | 230 | 86.95652174 | 250 | 700 | 2.8 | 880 | 500 | 1.76 |

TABLE 9-1
Operating parameters and UV spectrophotometry results of
nucleic acid samples from carrot tuber (continued)

| Nucleic acid | Nucleic acid | | | |
|---|---|---|---|---|
| sample No. | Type | OD260/280 | OD260/230 | Yield (μg) |
| J | RNA | 2.18 | 1.45 | 1.315 |

(2) Same as step (2) in Example 9.

Washed the white solid as in Example 9.

Added 50 μl of ice-bath high-purity water on the ice to dissolve the nucleic acid in the centrifuge tube, which is designated nucleic acid sample J for storage at −20° C. or detection.

UV spectrophotometry results: The assay was performed as in Example 1 and the results are shown in Table 9. The OD260/230 of nucleic acid sample J was less than 1.5, indicating the presence of particulate impurities. The solution of J was visibly not completely transparent.

Agarose gel electrophoresis: same as Example 1.

Agarose gel electrophoresis results: the electrophoresis profile was shown in FIG. 12, indicating that the 25s rRNA and 18s rRNA bands at the edge of nucleic acid sample J were clear, and the brightness ratio of the two exceeded 2.

Conclusion: The results of Example 8 showed that the present invention method could be used to extract intact RNA molecules from the carrot tuber, but the nucleic acid sample also contained impurities such as insoluble substances.

### Example 12 Purification of Contaminated RNA Samples

A method for isolating and purifying solid nucleic acid from biomaterials, including the following steps:

(1) At room temperature, 40 μl of nucleic acid sample J obtained from Example 11 and 60 μl of high-purity water were added to a 1.5 ml centrifuge tube and mixed to obtain a liquid containing naked RNA.

200 μl of precipitant (5 M NaCl aqueous solution) was added, shaken to mix, and centrifuged at 16,000 g for 0.5 min at RT. Transferred the supernatant to another centrifuge tube (see Table 10, including Tables 10-1 and 10-2).

TABLE 10-1
Operating parameters and UV spectrophotometry results of nucleic acid samples from contaminated RNA sample

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| K | 40 | 160 | — | 200 | 200 | 1 | 400 | 400 | 1 |

TABLE 10-2
Operating parameters and UV spectrophotometry results of nucleic
acid samples from contaminated RNA sample (continued)

| Nucleic acid | Nucleic acid | | | |
|---|---|---|---|---|
| sample No. | Type | OD260/280 | OD260/230 | Yield (μg) |
| K | RNA | 2.17 | 2.08 | 0.844 |

(2) Added 400 μl of isopropanol to the supernatant, mixed well, and left for 15 min at RT. After centrifugation at 12,000 g for 5 min at RT, the liquid in the centrifuge tube was decanted to obtain a white precipitate in the centrifuge tube.

Washed the white solid as in Example 9.

Added 40 μl of ultrapure water on the ice to dissolve the white solid in the centrifuge tube and obtained nucleic acid sample K, which was stored at −20° C. or detected.

UV spectrophotometry results: The assay was performed as in Example 1. The results are given in Table 10. The OD260/230 of nucleic acid sample K is greater than 2.0, indicating that there are no particulate insoluble substances in it—this is also confirmed by the transparency of the liquid in sample K observed with the naked eye.

Agarose gel electrophoresis: same as Example 1,

Agarose gel electrophoresis result: the electrophoretic pattern is shown in FIG. 12: nucleic acid sample K contained 28s rRNA and 18s rRNA bands with clear edges, indicating that the RNA molecule has not been degraded.

Conclusion: Example 12 demonstrates that the method of the present invention can easily remove impurities such as insoluble substances from RNA samples and ensure the integrity of RNA molecules.

### Example 13 Isolation and Purification of Solid RNA from Rosebud Petals

A method for isolating and purifying solid nucleic acids from biomaterials, comprising the following steps:

(1) 60 mg of rosebud petals and 1 ml of homogenization-dissociation reagent (prepared by the same method as in Example 2) were added to a 1 ml Dounce homogenizer and rapidly homogenized at RT to obtain a liquid containing naked RNA.

250 μl of the liquid was then transferred to a 1.50 ml centrifuge tube, giving a total of two tubes. 700 μl of precipitant (as in the precipitant in Example 1) was added to each tube and the tubes were inverted several times to mix the liquid. After centrifugation at 12,000 g for 5 min at RT, the supernatant was transferred to another centrifuge tube as shown in Table 11-1, Table 11-2.

TABLE 11-1
Operating parameters and UV spectrophotometry results of nucleic acid samples from rosebud petals

| | | | | | | Precipitant | | | |
|---|---|---|---|---|---|---|---|---|---|
| | | | Biomaterial | Naked | | Vol./ | | | |
| | | | mass/ | nucleic | | Naked | | | |
| Nucleic | | Homo- | Homo- | acid | | nucleic | | | Supermatant |
| acid | Biomaterial | Diss | Diss | liquid | Precipitant | acid | Supermatant | Isopropanol | vol./ |
| sample | mass | Vol. | Vol. | Vol. | Vol. | liquid | vol. | vol. | Isopropanol |
| No. | (mg) | (μl) | (mg/ml) | (μl) | (μl) | Vol. | (μl) | (μl) | vol. |
| L0 | 60 | 1000 | 60 | 250 | 700 | 2.8 | 880 | 500 | 1.76 |
| L1 | 60 | 1000 | 60 | 250 | 700 | 2.8 | 880 | 500 | 1.76 |

TABLE 11-2
Operating parameters and UV spectrophotometry results of
nucleic acid samples from rosebud petals (continued)

| Nucleic acid | Nucleic acid | | | |
|---|---|---|---|---|
| sample No. | Type | OD260/280 | OD260/230 | Yield (μg) |
| L0 | RNA | 1.97 | 2.24 | 4.99 |
| L1 | RNA | 1.97 | 2.24 | 4.488 |

(2) Same as step (2) in Example 9.

The white solid obtained in step (2) was washed as in Example 9.

50 μl of ice-bathed high-purity water was added to a centrifuge tube on ice and the solid nucleic acid in the tube was dissolved to obtain sample L0 for detection. Another solid nucleic acid was placed at RT for one month and its RIN (RNA integrity number) was determined by Beijing Nuohu Zhixuan Science and Technology Co., Ltd. using an Agilent 2100 Bioanalyzer (Agilent Technologies, Foster City CA). The result was referred to as sample L1.

UV spectrophotometry results: The assay was performed as in Example 1. The results for nucleic acid sample L1 were determined by Nuohu Zhixuan Co., Ltd. as shown in Table 11.

Agarose gel electrophoresis result: 1) the electrophoresis of nucleic acid sample L0 was the same as in Example 1. The electrophoresis of nucleic acid sample L1 was performed by Nuohu Zhixuan Biological Technology Co., Ltd.

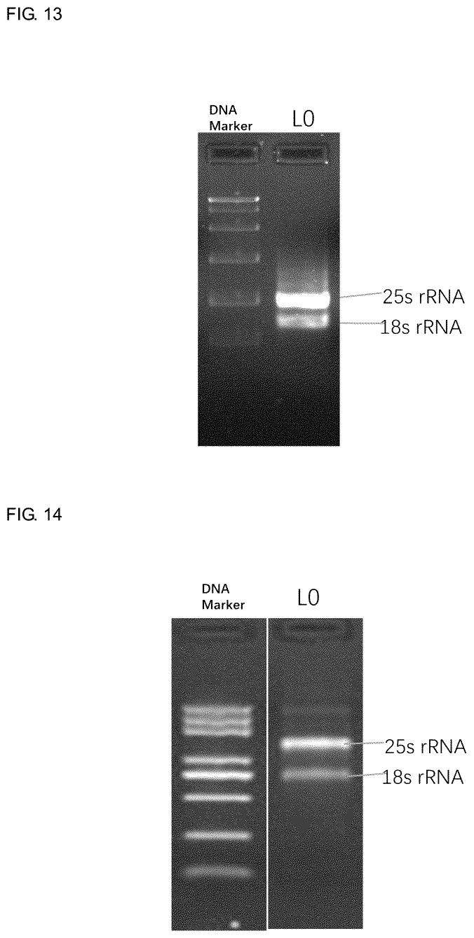

The electrophoresis results showed that 1) the electrophoretic pattern of nucleic acid sample L0, as shown in FIG. 13, indicated the presence of 18s rRNA and 25s rRNA bands, and the margins were complete, confirming that intact RNA molecules were extracted from rosebud petals; 2) the electrophoretic pattern of nucleic acid sample L1, as shown in FIG. 14, showed 18s rRNA and 25s rRNA bands with complete margins, indicating that the RNA molecules in the solid RNA extracted from rosebud petals could maintain their integrity after storage at RT for one month.

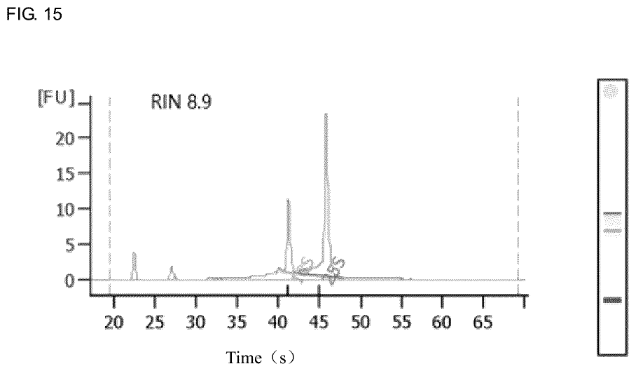

RIN value determination results: As shown in FIG. 15, nucleic acid sample L1 contained RNA, and its RIN value (RNA integrity number) was 8.9, which could meet all the requirements for RNA analysis.

Conclusion: The results of Example 13 demonstrate that the method of the present invention can be used to extract solid RNA from rosebud petals; the solid RNA can maintain the integrity and high purity of its RNA components even after being stored at RT for one month and can be used for various RNA detection, including RNA sequencing. This provides technical support for the separation, extraction, and detection of RNA, combined with the simplicity of the method, low toxicity of reagents, and low requirements for operating equipment and environment. This lays the foundation for the widespread application of RNA extraction and detection.

### Example 14 Two Methods for Extraction of Mouse Liver RNA and its Detection by Real-Time Fluorescent PCR

Operation of real-time fluorescence PCR

(1)Extraction of mouse liver RNA: C57 BL/6 mouse liver RNA was extracted using the Trizol method and the method implemented in Example 13, respectively, and different storage treatments were performed according to the method in Table 12.

(2) Reverse transcription of RNA samples: cDNA was synthesized using the SuperScript III RT Kit according to the instructions (ABI-Invitrogen) and used for qPCR analysis.

(3) Detection of mouse β-actin mRNA copy number by real-time fluorescence PCR: The amplification system (20 μl) contained 2 μl cDNA, 10 μl qPCR mix, 1 μl primer F (5′TATAAAACCCGGCGGCGGCGCA), 1 μl primer R (5′TCATCCATGGCGAACTGGTG) and 6 μl ddH2O. The qPCR reaction was performed on an ABI 7900 qPCR instrument under the following conditions 2 min incubation at 95° C.; followed by 40 cycles of 20 sec incubation at 94ºC, 20 sec incubation at 60° C. and 30 sec incubation at 72° C.

TABLE 12
Comparison of β-Actin mRNA Relative Copy Numbers in RNA Samples
Extracted by Two Methods from the Liver of C57 BL/6 Mice

| RNA | | | | | Relative |
|---|---|---|---|---|---|
| extraction | Storage conditions | | | Average | copy |
| method | Status | Temperature | Time | Ct | number* |
| Trizol method | Aqueous | No | No | 23.09 | 1 |
| | solution | | | | |
| This invention | Aqueous | −20° C. | 7 days | 21.53 | 2.95 |
| method | solution | | | | |
| | Solid | Room | 60 days | 22.15 | 1.92 |
| | | Temperature | | | |

Note:
*The relative copy number is calculated based on 2^{-ΔΔCt}.

### Real-Time Fluorescence PCR Results

(1) As shown in Table 12, according to the Livak method (Livak, K. J., and Schmittgen, T. D. (2001). Analysis of relative gene expression data using real-time quantitative PCR and the 2AACT method. Methods. 25:402-408.), it is concluded that the copy number of β-actin mRNA detected by the RNA samples extracted by the present invention is approximately two to three times greater than that of the RNA samples extracted by the Trizol method. This indicates that the reverse transcription efficiency of the RNA obtained by the method of the present invention is greater than that of the RNA obtained by the Trizol method.

(2) Table 12 also shows that the detected β-actin mRNA copy number of the solid RNA samples extracted by the present invention method and kept at RT for two months is still greater than that of the corresponding liquid RNA samples extracted by the Trizol method. Therefore, the solid RNA extracted by the present invention can be stored and shipped at RT, providing the possibility of temporal and spatial separation for RNA extraction and detection.

Conclusion: By using the solid RNA extracted by the present invention method and stored at RT for two months, the detection quality is better than that of the RNA solution currently extracted by the Trizol reagent, providing a feasible way for the easy storage and transportation of solid RNA and the spatiotemporal separation of RNA extraction and detection. It also lays the foundation for the large-scale application of RNA in the life sciences.

1. A method for isolating and purifying solid nucleic acids from biomaterials, comprising the following steps:
(1) mixing a liquid containing naked nucleic acids with an alkali metal salt solution with a precipitation effect at a volume ratio of 1:(1-11); centrifuging at room temperature, pouring the supernatant into a new centrifuge tube;
(2) conducting one of the following three ways:
way I:
adding isopropanol to the new centrifuge tube containing the supernatant obtained in step (1) at a volume ratio of (1-4.4):1, mixing well, standing at RT for 1-30 min, centrifuging, generating a white precipitate, discarding other than the white precipitate, washing so as to obtain solid RNA or a solid mixture of RNA and DNA for preservation;
way II:
adding isopropanol to the new centrifuge tube containing the supernatant obtained in step (1) at a volume ratio of (1-4.4):1, mixing well, standing at RT for 1-30 min, adding distilled water equivalent to (0.0714-0.1348) times the volume of the supernatant, mixing well, centrifuging, generating a white precipitate, discarding other than the white precipitate; washing so as to obtain solid DNA or a solid mixture of RNA and DNA for preservation;
way III:
1. adding isopropanol to the new centrifuge tube containing the supernatant obtained in step (1) at a volume ratio of (1.5-1.76):1, mixing well, standing at RT for 1-30 min, centrifuging, generating a white precipitate, pouring the liquid other than the white precipitate named the retention liquid into another new centrifuge tube; washing the white precipitate, obtaining solid RNA or a solid mixture of RNA and DNA for preservation;
2) adding distilled water to the new centrifuge tube containing the retention liquid, mixing well, standing at RT for 1-30 min, centrifuging, generating a white precipitate, discarding other than the white precipitate, washing so as to obtain solid DNAs for preservation; the ratio of the supernatant to distilled water is 1:(0.125-0.1705).

2. The method according to claim 1, wherein the liquid containing naked nucleic acid is prepared by following:
homogenizing 16.27-162.8 mg of biomaterial with 1 ml of homogenization-dissociation reagent in proportion, to obtain a liquid containing naked nucleic acid;
wherein, the homogenization-dissociation reagent is composed of formamide, an aqueous solution of alkali metal salts with a concentration of 5M-14M, and a compound for removal of secondary cellular metabolites in the ratio of 200 ml: 10-50 ml: 0-10 g.

3. The method according to claim 2, wherein the biomaterial is animal organ, animal tissue, animal cell, plant organ, plant tissue, plant cell, fungi, or bacteria.

4. The method according to claim 2, wherein the homogenizing-dissociating agent is composed of formamide in the ratio of 200 ml: 10-50 ml:2-5 g, an aqueous solution of alkali metal salts in the concentration of 5 M-14 M, and a compound for removing cellular secondary metabolites.

5. The method according to claim 1, wherein the alkali metal salt is lithium chloride or sodium chloride.

6. The method according to claim 1, wherein the washing is conducted by washing with a volume concentration of 70%-90% ethanol-water solution centrifuging at 2000-16000 g for 10-60s at RT, pouring off the washing solution, washing with absolute ethanol, and air-drying.

7. The method according to claim 2, wherein the compound that removes secondary metabolites of cells comprises at least one of casein, polyvinylpyrrolidone 40, and cetyltrimethylammonium bromide.

8. The method according to claim 2, wherein the alkali metal salt is lithium chloride or sodium chloride.

9. The method according to claim 4, wherein the alkali metal salt is lithium chloride or sodium chloride.

10. The method according to claim 4, wherein the compound that removes secondary metabolites of cells comprises at least one of casein, polyvinylpyrrolidone 40, and cetyltrimethylammonium bromide.
